(19) United States
(12) Patent Application Publication
(10) Pub. No.: US 2004/0265947 A1
(43) Pub. Date: Dec. 30, 2004

(54) Method for detecting bacteria culture under anaerobic conditions

(76) Inventors: Alain Rambach, Paris (FR); Christine Favier, Alennes-Les-Marais (FR)

Correspondence Address: BLAKELY SOKOLOFF TAYLOR & ZAFMAN, 12400 WILSHIRE BOULEVARD, SEVENTH FLOOR, LOS ANGELES, CA, 90025-1030, US

(21) Appl. No.: 10/806,965

(22) Filed: Mar. 22, 2004

Related U.S. Application Data
(62) Division of application No. 09/890,841, filed on Aug. 2, 2001.
Application No. 09/890,841 is a 371 of international application No. PCT/FR00/00241, filed on Feb. 2, 2000.

(30) Foreign Application Priority Data
Feb. 3, 1999 (FR) 99/01226

(51) Int. Cl. C12Q 1/18; C12Q 1/04

(52) U.S. Cl. 435/34

(57) ABSTRACT

A combination product for the detection of bacteria. The product includes at least one oxidizing metal complex and at least one substrate containing an indoxyl derivative resulting in an insoluble colored compound for use simultaneously, separately or spread out over time.

The present invention relates to a bacterial culture medium, for use under anaerobic conditions, comprising at least one metal complex which allows the oxidative polymerization of an indoxyl derivative and a substrate containing an indoxyl derivative resulting in an insoluble colored compound. Said metal complex, in particular ammoniacal iron citrate, has a concentration of between 0.3 and 0.9 mg/ml, preferably 0.6 mg/ml. Advantageously, the culture medium according to the invention may comprise a substrate such as X-Gal, at a concentration of between 10 and 500 mg/l.

Numerous methods for the identification and counting of bacteria strains have been developed in order to satisfy the needs for diagnostic tests or tools in all technical and scientific fields relating to microbiology, in particular medicine and the agri-industries.

Such methods may prove extremely useful for the diagnosis of opportunistic infections whose symptoms are sometimes not very characteristic of the exact cause of the disease. For example, Crohn's disease. (CD) is a chronic inflammatory disease of the digestive tube. It manifests itself by abdominal pain, diarrhea, fever and undernourishment. The lesions are characterized by impairment of the digestion wall which is inflamed, thickened and ulcerated. This disease lasts for life, during which the patients undergo evolutive paroxysms followed by periods of remission.

The studies devoted to the modifications of the flora during CD have given conflicting results. However, most of them agree in concluding to an increase in the number of E. coli and Bacteroides of the group fragilis. No potentially pathogenic strain has been able to be distinguished as being distinct.

The study of a plasma glycoprotein secreted in stools, the α-1-proteinase inhibitor, has demonstrated an impairment in the bacterial metabolism in patients suffering from CD. Indeed, in healthy subjects, this glycoprotein is deglycosylated along the whole length of the colon following the action of exoglycosidases of bacterial origin. On the other hand, in patients, it remains glycosylated, which results in a defect in the activity of these osidases. This defect has indeed been proven by assays of glycosidase activities in fecal extracts. The enzymatic activities, and in particular that of β-galactosidase, were found to be considerably reduced in the patients compared with the controls, Favier et al., (1996), Differenciation and identification of human fecal anaerobic bacteria producing β-galactosidase (a new methodology), Journal of Microgiological Methods 27, 25-31; Favier et al., (1997), Fecal β-D-galactosidase Production and Bifidobacteria Are Decreased in Crohn's Disease, Digestive Diseases and Sciences, 42, 817-822.

The capacity of the fecal flora in these patients and in healthy subjects, incubated under appropriate conditions, to produce and to liberate β-galactosidase, was studied. To do this, fecal samples are cultured, under an anaerobic atmosphere and at 37° C., in a Wilkins Chalgren (WC) broth supplemented with pig gastric mucins (in order to promote the growth of microorganisms and the production of exoglycosidases). The β-galactosidase activity is added on the supernatants of aliquots collected at the beginning (2 h) and at the end (22 h) of incubation.

Thus, a methodology which makes it possible to selectively count the anaerobic microorganisms releasing β-galactosidase in the feces was developed.

However, given the complexity of the flora, the conventionally used methods, which consist in isolating the colonies, identifying the microorganisms and then assaying the enzymatic activity which they produce do not appear to be capable of responding to the problem posed.

Recently, the use of chromogenic substrates such as 5-bromo-4-chloro-3-indolyl-β-D-galactopyranoside or X-gal, has allowed the detection of Lac⁺ anaerobic microorganisms. Following enzymatic hydrolysis, the substrate undergoes oxidative polymerization which causes the formation of a blue precipitate. This methodology, which makes it possible to differentiate, directly on a Petri dish, the microorganisms capable of releasing β-gal in the medium, was applied to the study of fecal microorganisms in patients and healthy subjects. The results obtained were compared with those from the analysis, with the aid of selective media, of the principal groups of microorganisms known for their capacity to produce β-galactosidase. Bacteroides, Lactobacillus and Bifidobacterium.

Examples of such media are presented in Chevalier, P., Roy, D. and Savoie, L., (1991) X-Gal based medium for simultaneous enumeration of bifidobacteria and lactic acid bacteria in milk, J. Microbiol. Methods 13, 75-83; and in Livingston S J, Kominos S D, Lee R B, (1978), New medium for selection and presumptive identification of the Bacteroides fragilis group, J. Clin, Microbiol 7, 448-453.

However, it is sometimes difficult to visualize the bacteria. There has been developed, in the context of the present invention, a medium containing an oxidizing metal complex which makes it possible in particular to intensify the halos of colors obtained around the colonies. This improvement in the technique mentioned above has been achieved so as to promote the oxidative reaction of the substrate in the reduced medium necessary for the growth of bacteria under anaerobic conditions.

Thus, no prior art document describes or suggests the present invention as defined hereinafter.

### DESCRIPTION

The present invention relates to a bacterial culture medium, for use under anaerobic conditions, comprising at least one metal complex which allows the oxidative polymerization of an indoxyl derivative and a substrate containing an indoxyl derivative resulting in an insoluble colored compound. Said metal complex, in particular ammoniacal iron citrate, has a concentration of between 0.3 and 0.9 mg/ml, preferably 0.6 mg/ml. The culture medium according to the invention may comprise at least one selected from X-Gal, X-Phos, X-acglmn, Mag-Gal, Mag-α-Gal, and Mag-Phos, preferably X-Gal, at a concentration of between 10 and 500 mg/l, particularly between 50 and 200 mg/l, preferably at 100 mg/ml.

The expression “bacterium” in the context of the invention is understood to mean anaerobic bacteria, aerobic anaerobic bacteria, and any bacterium producing, naturally or otherwise, a β-galactosidase. Among the transformed bacteria, there may be mentioned in particular a bacterium transformed by a plasmid containing the LacZ gene, optionally under the control of a promoter of interest.

Consequently, the medium according to the invention is intended for the detection of anaerobic bacteria, aerobic anaerobic bacteria and any bacterium producing a β-galactosidase.

There may be mentioned, by way of example, bacteria of the genus Bifidobacterium, Clostridium, Citrobacter, Escherichia, and/or Bacteroides, in particular of the strains Bifidobacterium bifidum, Clostridium perfringens, Clostridium butyricum, E. coli, and/or Bacteroides fragilis.

Preferably, this culture medium comprises cysteinated Columbia medium well known to a person skilled in the art, whose ingredients and characteristics are the following (as a base qsp according to the manufacturer):

| | | |
|---|---|---|
| Glucose | 5 | g |
| Cysteine hydrochlorate | 0.3 | g |
| Agar | 5 | g |
| Water | 1000 | ml |
| PH | 7.3 | |
| Autoclaving | 15 | mm, 120° C. |

However, the medium according to the invention is not limited to a list of particular ingredients, such that it can be adapted to the culture of a given bacterium which it is sought to detect. For example, the TSC medium, described below, can serve as a base for the preparation of the medium according to the invention.

TSC medium (base qsp according to the manufacturer):

| | | |
|---|---|---|
| Tryptose | 15 | g |
| Soya bean flour peptone | 5 | g |
| Yeast extract | 5 | g |
| Sodium disulfite | 1 | g |
| Agar | 15 | g |
| Water | 1000 | ml |

The culture medium according to the invention may contain, in addition, magnesium sulfate at a concentration of between 5 mM and 100 mM, preferably 20 mM, and/or at least one antibiotic, for example cycloserine, preferably at 0.4 g/l, neomycin supplemented with polymyxin, preferably at 0.02 g/l and 0.05 g/l respectively.

An additional aspect of the present invention relates to a combination product comprising at least one oxidizing metal complex and at least one substrate containing an indoxyl derivative resulting in an insoluble colored compound for use simultaneously, separately or spread out over time, intended for the detection of bacteria. Said substrate may be selected from X-Gal, X-Phos, X-acglmn, Mag-Gal, Mag-α-Gal, and Mag-Phos, preferably X-Gal, and said metal complex is ammoniacal iron citrate.

This combination product is characterized in that the metal complex and the substrate are carried in an aqueous solvent at a concentration of between 3 and 900 mg/ml, preferably at 60 mg/ml, or an organic solvent at a concentration of between 100 mg/l and 50 g/l, particularly between 500 mg/l and 20 g/l, preferably at 10 g/l. The combination product according to the invention may contain, in addition, magnesium sulfate at a concentration of between 50 mM and 10 M, preferably 2 M, and/or at least one antibiotic.

The subject of an advantageous aspect of the present invention is a bacterial detection kit comprising a combination product as defined above.

In the context of the invention, the term “detection” is understood to mean the visualization, optionally the identification, and the quantification of bacteria.

The present invention also relates to a method for the detection of bacteria, characterized in that it comprises the following steps:

a) there are added to a medium which may contain said bacteria, cultured under anaerobic conditions, at least one substrate containing an indoxyl derivative resulting in an insoluble colored compound,

b) at least one oxidizing metal complex, in particular ammoniacal iron citrate, is added,

c) the appearance of a colored precipitate around the colonies (halo) and/or a color of the colonies is visualized.

In another embodiment, the method for the detection of bacteria comprises the following steps:

a) said bacteria are cultured in a medium according to the invention under anaerobic conditions,

b) the appearance of a colored precipitate around the colonies (halo) and/or a color of the colonies is visualized;

or alternatively the following steps:

a) a combination product according to the invention is added to a medium which may contain said bacteria cultured under anaerobic conditions,

b) the appearance of a colored precipitate around the colonies (halo) and/or a color of the colonies is visualized.

An additional aspect of the present invention relates to the use of an oxidizing metal complex, preferably ammoniacal iron citrate, for catalyzing the oxidative polymerization of indoxyl derivatives resulting in an insoluble colored compound, in particular for improving the detection of the release of an indoxyl derivative by an enzyme from a substrate containing an indoxyl derivative, it being possible for said substrate to be a substrate selected from X-Gal, X-Phos, X-acglmn, Mag-Gal, Mag-α-Gal, and Mag-Phos, preferably X-Gal. Said metal complex makes it possible to intensify the colored halo and/or to increase the color of the colonies. Indeed, it reacts with the indoxyl derivative according to the invention to give a colored compound which precipitates.

The invention also relates to the use of a medium, of a combination product or of a kit as described above for the detection of bacteria which possess an enzyme allowing the release of an indoxyl derivative from a substrate containing an indoxyl derivative.

The addition of ammoniacal iron citrate makes it possible, not only to visualize colors which do not appear in culture in jars under anaerobic conditions, but also to intensify the halo of colors of the colonies cultured under a plastic bag under anaerobic conditions. The columbia medium appears to be completely advantageous for the appearance of the colors. Of course, the colors and the intensity of the colors obtained depend on their strains, the level of expression and of secretion of β-galactosidase.

The examples below are given to illustrate the present invention but they do not limit the modalities of implementation thereof.

### EXAMPLE 1

### Medium Intended for the Detection of Bifidobacteria

The capacity of ammoniacal iron citrate (AIC) to increase the formation of indigo in a Generbag Anaer® bag and in a jar was determined.

A strain of Bifidobacterium bifidum was inoculated into the cysteinated Columbia medium+X-Gal with or without AIC (0.3 g/l), and then the effect of the addition of AIC was tested before or after autoclaving/regeneration.

The colonies in the Generbag Anaer® bag are surrounded by a halo which is more intense in the presence of AIC, and the halos became visible in a jar.

The bacterial count shows a reduction in the number of microorganisms when. AIC is added to the medium after autoclaving/regeneration.

Thus, AIC, added before regeneration of the bottle, promotes the appearance of the halo. Various concentrations of AIC were then tested on three Bifidobacterium strains. The results are presented in table I below.

TABLE I

| AIC (g/l) | 0 | 0.3 | 0.6 | 0.9 |
|---|---|---|---|---|
| B. bifidum | ø | + | ++ | +++↓ |
| B. longum | ø | + | ++ | ++ |
| B. dentium | ø | + | ++ | ++ |

ø: without halo
+ → +++: intensity of the halo
↓: reduction in the microorganisms (here by 90%)

Consequently, 0.6 g/l of AIC appears to be an ideal concentration for visualizing the presence of a halo around the Bifidobacterium colonies after culturing in a jar.

### EXAMPLE 2

### Study of Various Substrates in Order to Cause the Color to Appear Under Anaerobic Conditions by Addition of AIC

Various substrates were tested (100 mg/l) in cysteinated Columbia medium, in the presence and in the absence of AIC (0.6 g/l).

2.1 Medium Supplemented with X-Gal

The presence of AIC made it possible to observe the color due to the hydrolysis of the X-Gal substrate for the Clostridium colonies:

The colonies of C. perfringens (β-galactosidase+) are ocher without AIC; green-blue with a blue halo in the presence of AIC. The colonies of C. butyricum ((β-galactosidase+) are cream-colored without AIC; greenish cream-colored surrounded by a slight green-blue halo with AIC. On the other hand, the colonies of Citrobacter ((β-galactosidase+) remain cream-colored with or without AIC: either the AIC is not sufficient in order to see the color, or the microorganisms did not hydrolyze the substrate. In this strain, the LacZ gene is undoubtedly under the control of the lactose operon. Consequently, it is necessary to add lactose to the culture medium in order to induce the expression of β-galactosidase.

The differences between the medium with AIC and the medium without the AIC are particularly great for the strains of C. perfringens and C. butyricum.

2.2 Medium Supplemented with Mag-Gal

The colonies of C. perfringens ((β-galactosidase+) are ocher without AIC; pink with a pink halo in the presence of AIC.

The colonies of C. butyricum (β-galactosidase+) are cream-colored without AIC; pink with a pink halo with AIC, and those of Citrobacter (β-galactosidase+) are cream-colored without AIC; pink with AIC. The presence of AIC made it possible to observe the coloring due to the hydrolysis of the Mag-Gal substrate for the colonies of Clostridium butyricum and for Citrobacter. The enzymes of the latter would therefore not be inducible but would be more capable of hydrolzing Mag-Gal than X-Gal.

The differences between the medium with AIC and the medium without AIC are, in order, greatly marked for the strain of: C. perfringens, C. butyricum, Citrobacter.

2.3 Medium Supplemented with X-Phos

The colonies of C. perfringens (alkaline phosphatase+) are cream-colored without AIC; greenish cream-colored with a slight blue halo in the presence of AIC.

The colonies of C. butyricum (alkaline phosphatase−) are cream-colored without AIC; cream-colored with a very light halo near the colony and then blue at the periphery with AIC, and those of Citrobacter are cream-colored without AIC, green-blue with a light blue-green halo with AIC.

The colonies of E. coli are very light greenish cream-colored without AIC and darker with AIC. Finally, those of Bacteroides fragilis (alkaline phosphatase+) are cream-colored when they are isolated, blue in group without AIC; deep cream-colored, light brown with AIC.

The differences between the medium with AIC and the medium without AIC are very marked for the strain of: Citrobacter, then there are in order: C. butyricum, C. perfringens, E. coli and finally B. fragilis. Even if the colors are not sharp, the presence of AIC made it possible to observe the color (C. perfringens, C. butyricum), or to increase the color (E. coli) due to the hydrolysis of the X-Phos substrate. As regards B. fragilis, this releases the extra cellular enzymes which form halos of undefinable colors.

2.4 Medium Supplemented with Mag-Phos

The colonies of C. perfringens (alkaline phosphatase+) are cream-colored without AIC; pink with a pink halo in the presence of AIC. Those of C. butyricum (alkaline phosphatase−) are cream-colored without AIC; pink with a pink halo with AIC. Those of Citrobacter are cream-colored. (darker center) without AIC; pink (pink agar) with AIC.

Those of E. coli are cream-colored (darker center) without AIC; pink (pink agar) with AIC. That of Bacteroides fragilis are cream-pink without AIC; cream-pink with a brownish halo with AIC.

The presence of AIC made it possible to observe the color due to the hydrolysis of the Mag-Phos substrate for the colonies of Clostridium, E. coli and Citrobacter.

The differences between the medium with AIC and the medium without AIC are more marked for the strains of: C. perfringens and C. butyricium, then there are Citrobacter and E. coli, and finally B. fragilis.

2.5 Medium Supplemented with Mag-α-Gal

The colonies of C. perfringens (Mag-α-Gal+) are cream-colored without AIC; pink in the presence of AIC. Those of Bacteroides fragilis (Mag-α-Gal+) are dark cream-colored without AIC; cream-pink with AIC. Those of C. butyricum (Mag-α-Gal+), Citrobacter and E. coli are cream-colored without AIC; darker or light cream-colored with AIC.

The differences between the medium with AIC and the medium without AIC are more marked for the strains of: C. perfringens and B. fragilis, then there are C. butyricum, Citrobacter and E. coli.

2.6 Medium Supplemented with X-acglmn

The colonies of C. perfringens (X-acglmn+) are cream-colored without AIC; very slightly greenish cream-colored in the presence of AIC. Those of C. butyricum (X-acglmn−) do not exhibit growth without AIC; are greenish with a blue halo in the presence of AIC. Bacteroides fragilis (X-acglmn+), Citrobacter and E. coli are cream-colored with or without AIC.

The differences between the medium with AIC and the medium without AIC are more marked for the strains of: C. perfringens.

In conclusion, it is evident from these studies that the colors appeared in a medium supplemented with AIC, for the majority of the colonies. C. butyricum gives results which contradict its presumed enzymatic activities; however, the strain used is not necessarily represented here of the species.

### EXAMPLE 3

### Experiments in “Normal” TSC Medium

To be as close as possible to the selective media allowing enumeration of Clostridii, a first study was carried out in “normal” basic TSC medium with disulfite and AIC (1.0 g/l), and without antibiotics.

The colonies of Clostridium perfringens are black. Whether X-Gal, Mag-Phos, X-glu or X-glucu is added, the colors due to the hydrolysis of these substrates remain difficult to see. Nevertheless, the blue-gray halos around the colonies of C. perfringens in the presence of X-Gal (100 mg/l, combined with Mag-Phos 50 m/l or with X-glucu 100 m/l) can make it possible to distinguish between the C. perfringens and the other microorganisms. These halos are also observed around colonies of E. coli which are all blue (solely as X-Gal+ Mag-Phos).

Thus, the appearance of the color is impeded by the use of disulfites by C. perfringens. It appears necessary to work in a medium without disulfite.

### EXAMPLE 4

### Experiments in TSC Medium Without Disulfite

The substrates X-Gal, Mag-Gal, X-Phos and Mag-Phos (100 mg/l) were tested in the presence and in the absence of AIC (0.6 g/l) by reusing the base of the TSC medium (TSC medium without antibiotic, and this time without disulfite). The colors are in general less sharp than in Columbia medium.

With X-Gal, the differences between the medium with AIC and the medium without AIC are more marked for the strains of: C. perfringens and E. coli, and then Citrobacter and B. fragilis. C. butyricum remains cream-colored with AIC.

With Mag-Gal, the differences between the medium with AIC and the medium without AIC are more marked for the strain of: C. perfringens and then for those of E. coli and Citrobacter, and finally B. fragilis. C. butyricum did not grow in the absence of AIC.

With X-Phos, the differences between the medium with AIC and the medium without AIC are more marked for the strain of: E. coli, Citrobacter and then those of C. perfringens and B. fragilis. C. butyricum did not grow in the absence of AIC.

With Mag-Phos, the colonies are just darker in the presence of AIC, the differences between the medium with AIC and the medium without AIC are more marked than the strains of E. coli and Citrobacter and then for that of C. perfringens.

In conclusion, it is preferable to use the cysteinated Columbia medium to cause a more intense color to appear compared with the TSC medium.

If a value is given for the intensity of the colors of the colonies according to the substrate, β-galactosidase appears to make it possible to visualize better C. perfringens compared with phosphatase (see table II below)

TABLE II

| | C. perfringens | C. butyricum | Citrobacter | E. coli | B. fragilis |
|---|---|---|---|---|---|
| X-Gal | 3 | 2 | 0 | Ø | Ø |
| X-Gal TSC | 3 | 0 | 2 | 3 | 2 |
| Mag-Gal | 3 | 2 | 1 | Ø | Ø |
| Mag-Gal TSC | 3 | Ø | 2 | 2 | 1 |
| GALACTOSIDASE | 3 | 1.33 | 1.25 | 2.5 | 1.5 |
| X-Phos | 2 | 2 | 3 | 2 | 1 |
| X-Phos TSC | 2 | Ø | 3 | 3 | 2 |
| Mag-Phos | 3 | 3 | 2 | 2 | 1 |
| Mag-Phos TSC | 2 | 0 | 3 | 3 | 0 |
| PHOSPHATASE | 2.25 | 1.66 | 2.75 | 2.5 | 1 |

Ø: lack of data
3 to 0: relative intensity of the color of the colonies for a given medium with a given substrate

### EXAMPLE 5

### Study of the Efficacy of the Addition of Ferricyanides to Cause the Color to Appear Under Anaerobic Conditions

Ferricyanide was used alone at 0.6 g/l. There is no difference between the media containing these products or otherwise for the substrate X-Gal.

Only the colonies of Bacteroides fragilis are blue with X-Phos in the presence of ferricyanide.

Thus, X-phos+ferricyanide may be an excellent medium for preidentifying Bacteroides.

### EXAMPLE 6

### Improvement of the Medium Intended for the Study of Clostridii by Addition of Antibiotics

To resemble the TSC medium for selecting C. perfringens, antibiotics were added to the TSC base:

either cycloserine (0.4 g/l);

or neomycin supplemented with polymyxin (0.02 and 0.05 g/l respectively);

or the three together.

All the colonies (C. perfringens, C. butyricum, E. coli, B. fragilis, Citrobacter) grow in the presence of cycloserine alone. On the other hand, the combination neomycin and polymyxin (0.02 g/l and 0.05 g/l) makes it possible to inhibit the growth of B. fragilis, E. Coli and Citrobacter.

The following two strains of Clostridium grow:

the colonies of C. butyricum remain cream-colored, those of C. perfringens are slightly colored (colonies having a pink center with Mag-Gal (100 mg/l) and AIC (0.6 g/l) and colonies having a greenish center surrounded by a very slight halo with X-Gal (100 mg/l) and AIC (0.6 g/l)).

In cysteinated Columbia medium, the colors ought to be darker and the addition of antibiotics, which possess the great advantage of being autoclavable, ought to make it possible to inhibit the other microrganisms.

The growth of C. butyricum is not disruptive in TSC medium given that the colonies remain cream-colored, but will be disruptive in Columbia medium because the colonies have colors close to those of C. perfringens.

1. A combination product comprising at least one oxidizing metal complex and at least one substrate containing an indoxyl derivative resulting in an insoluble colored compound for use simultaneously, separately or spread out over time, intended for the detection of bacteria:

2. The combination product as claimed in claim 1, wherein said substrate is selected from X-Gal, X-Phos, X-acglmn, Mag-Gal, Mag-α-Gal, and Mag-Phos, preferably X-Gal.

3. The combination product as claimed in claim 1, wherein said metal complex is ammoniacal iron citrate.

4 The combination product as claimed in claim 1, wherein said metal complex and said substrate are carried in an aqueous solvent at a concentration of between 3 and 900 mg/ml, preferably at 60 mg/ml, or an organic solvent at a concentration of between 100 mg/l and 50 g/l, particularly between 500 mg/l and 20 g/l, preferably at 10 g/l.

5. The combination product as claimed in claim 1, further comprising magnesium sulfate at a concentration of between 50 mM and 10 M, preferably 2 M, and/or at least one antibiotic.

6. A bacterial detection kit comprising a combination product as claimed in claim 1.

7. A method for the detection of bacteria, comprising the following steps:
a) a combination product as claimed in claim 1 added to a medium which may contain said bacteria cultured under anaerobic conditions,
b) the appearance of a colored precipitate around the colonies (halo) and/or a color of the colonies is visualized.

8. The use of an oxidizing metal complex for catalyzing the oxidative polymerization of indoxyl derivatives resulting in an insoluble colored compound.

9. The use as claimed in claim 8, for improving the detection of the release of an indoxyl derivative by an enzyme from a substrate containing an indoxyl derivative, it being possible for said substrate to be a substrate selected from X-Gal, X-Phos, X-acglmn, Mag-Gal, Mag-α-Gal, and Mag-Phos, preferably X-Gal.

10. The use as claimed in claim 9, for intensifying the colored halo and/or for increasing the color of the colonies.

11. The use as claimed in claim 8, wherein the oxidizing metal complex is ammoniacal iron citrate.

12. The use of a combination product as claimed in claim 1, for the detection of bacteria which possess an enzyme allowing the release of an indoxyl derivative from a substrate containing an indoxyl derivative.
